(19) United States
(12) Patent Application Publication
(10) Pub. No.: US 2020/0306026 A1
(43) Pub. Date: Oct. 1, 2020

(54) FILTER DEVICE

(71) Applicants: Dai YAMANOUCHI, Madison, WI (US); KAWASUMI LABORATORIES, INC., Saiki-shi (JP)

(72) Inventors: Dai YAMANOUCHI, Madison, WI (US); Takashi Yoshimori, Bungo-ono-shi (JP)

(73) Assignees: Dai YAMANOUCHI, Madison, WI (US); KAWASUMI LABORATORIES, INC., Saiki-shi (JP)

(21) Appl. No.: 16/821,284

(22) Filed: Mar. 17, 2020

Related U.S. Application Data
(60) Provisional application No. 62/823,236, filed on Mar. 25, 2019.

(51) Int. Cl. A61F 2/01

(52) U.S. Cl. CPC A61F 2/01; A61B 17/221; A61F 2002/011

(57) ABSTRACT

An infarction preventive device includes a cylindrical filter, a tube configured to hold a base end portion of the filter and place the filter in a blood vessel, and a recovery member configured to recover the filter placed in the blood vessel into the tube.

### CROSS REFERENCE TO RELATED APPLICATION

This application claims benefit to U.S. Provisional Application No. 62/823,236 filed on Mar. 25, 2019, the entire content of which is incorporated herein by reference.

### TECHNICAL FIELD

The present invention relates to a filter device.

### BACKGROUND ART

Conventionally, there has been a concern that foreign substances such as thromboses may be generated and scattered in blood vessels during intravascular treatment, resulting in infarction. A related art vascular protection device having a filter which is attached to the tip of an elongated member inserted into a blood vessel. The filter captures foreign substances such as thromboses to prevent the foreign substances from entering terminal arteries, terminal veins, and the like. See, e.g., U.S. Pat. No. 8,182,507.

### SUMMARY

Incidentally, also when an aortic aneurysm, an aortic dissection, or the like generated in an aorta is treated using a stent graft, infarction may occur due to foreign substances such as generated thromboses or the like, and therefore it is preferable to prevent infarction using the aforementioned filter.

Herein, it is preferable to take a double structure that a shaft connected to the filter is inserted through a hollow portion of a sheath, from the viewpoint of ease in placing the filter and in recovering the placed filter. However, if an infarction preventive device is increased in diameter due to such a double structure, a burden on a patient during an operation may increase.

The present invention provides a filter device which can be decreased in diameter.

A filter device according to an aspect of the present invention includes a filter in a cylindrical form, a tube configured to hold a base end portion of the filter and place the filter in a body lumen, and a recovery member configured to recover the filter placed in the body lumen into the tube.

### BRIEF DESCRIPTION OF THE DRAWINGS

FIG. 1 is a schematic diagram of a state where a filter of an infarction preventive device according to the present invention is temporarily placed in a brachiocephalic artery;

FIG. 2 is a schematic diagram of a state where a filter of an infarction preventive device according to an embodiment of the present invention is placed at a predetermined position in a blood vessel;

FIG. 3 is a perspective view of the filter of the infarction preventive device according to the embodiment of the present invention;

FIG. 4A and FIG. 4B are perspective views of the infarction preventive device according to the embodiment of the present invention, FIG. 4A illustrating a region around an end portion on a distal end side of the filter in an enlarged manner, and FIG. 4B illustrating a region around an end portion on a base end side of the filter in an enlarged and partially broken manner;

FIG. 5A to FIG. 5D are cross-sectional views for explaining a procedure for housing the filter of the infarction preventive device according to the embodiment of the present invention in a sheath tube;

FIG. 6 is a perspective view of a filter of an infarction preventive device according to another embodiment of the present invention; and

FIG. 7 is a perspective view of a filter of an infarction preventive device according to another embodiment of the present invention.

### DETAILED DESCRIPTION

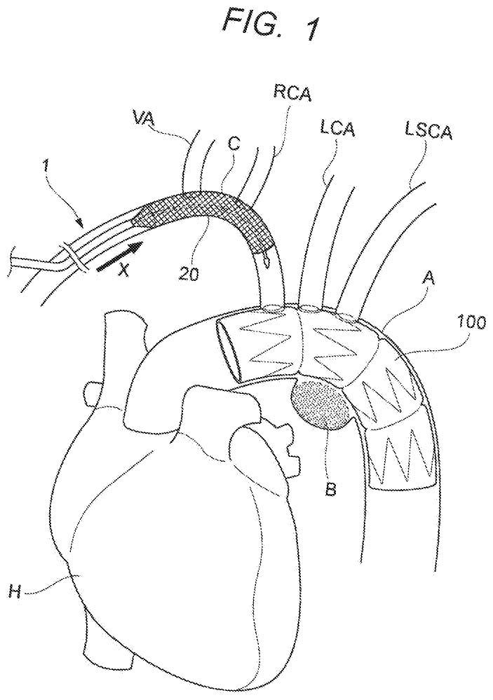

Hereinafter, specific embodiments of the infarction preventive device according to the present invention will be explained in detail with reference to the figures. First, outlines of a stent graft used for treatment of an aortic aneurysm or the like, and an infarction preventive device used in combination with the stent graft in the treatment will be explained with reference to FIG. 1.

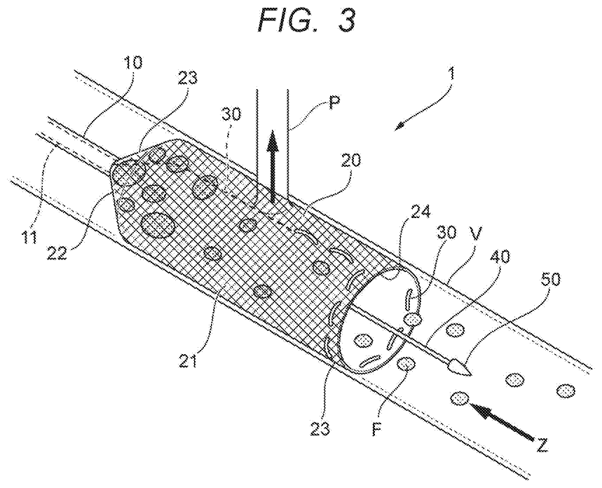

For example, in treatment for placing a stent graft 100 in an aortic arch A having an aortic aneurysm B, there are concerns that foreign substances F (see FIG. 3 and the like) such as thrombi are generated from the aortic arch A (source region) and scattered, and an infarction may occur in a blood vessel V different from the aorta. Thus, for example, as illustrated in FIG. 1, a filter 20 of an infarction preventive device 1 according to the present invention is previously and temporarily placed in a brachiocephalic artery C or e like before the stent graft 100 is placed. Specifically, the filter 20 is placed in the brachiocephalic artery C corresponding to a right common carotid artery (RCA) and a vertebral artery (VA).

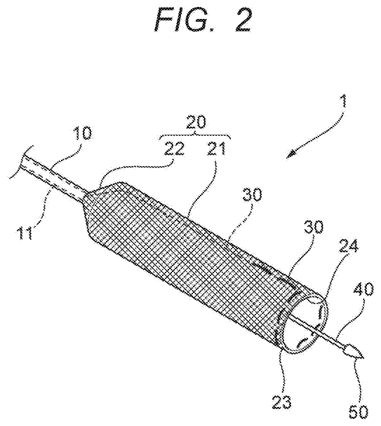

As illustrated in FIG. 2, the infarction preventive device 1 includes a hollow sheath tube 10, the filter 20 attached to a distal end of the sheath tube 10, a linear member 30 attached to the filter 20, a guide tube 40 disposed inside the filter 20, and a tip 50 attached to a distal end of the guide tube 40. Incidentally, during placement of the filter 20, a part on a distal end side of the filter 20 is located on a central side of the blood vessel V, and a part on a base end side of the filter 20 is located on a peripheral side of the blood vessel V.

The sheath tube 10 is made of any of various materials, such as a resin (plastic, elastomer or the like) and metal, having appropriate hardness and flexibility. An operator operates a manipulator (not illustrated) disposed outside a human body, so that the sheath tube 10 can move in the blood vessel V.

In addition, the sheath tube 10 can house the filter 20 in a hollow portion 11 of the sheath tube 10 such that the filter 20 is decreased in diameter. Subsequently, after the sheath tube 10 moves to a desired placement site, the filter 20 is released from the sheath tube 10, and thereby the filter 20 is placed so as to be increased in diameter.

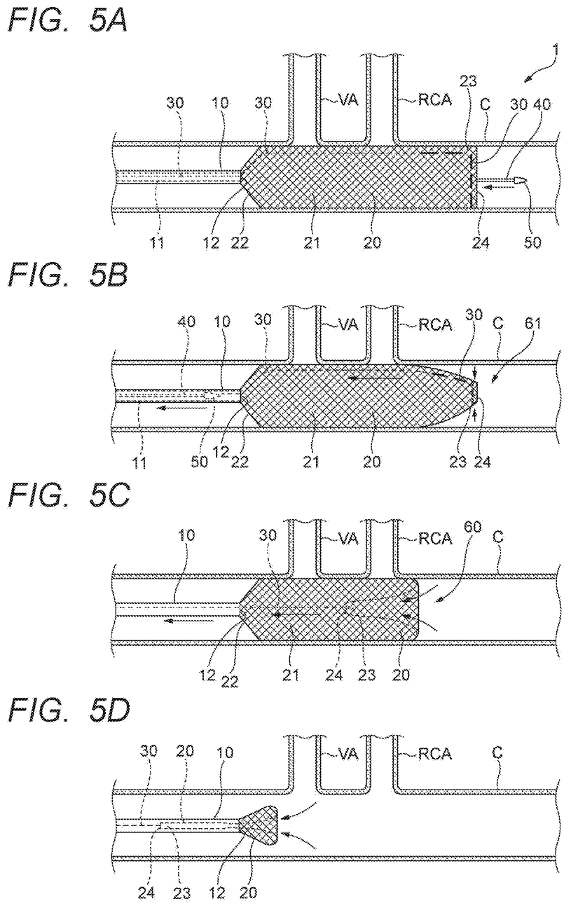

Incidentally, examples of the state of housing the filter 20 in the sheath tube 10 includes a state where the filter 20 is recovered from the blood vessel V described below (see FIG. 5D and the like), as well as a state where the filter 20 is reversed with an inner peripheral face out and an outer peripheral face in, and the like. However, the present invention is not limited to these states, and the state can be optionally changed as appropriate.

The filter 20 is placed in the blood vessel V such as the brachiocephalic artery C which is different from the aortic arch A (FIG. 1). In addition, the filter 20 includes a cylindrical main body portion 21 which is expandable and contractable as a whole and has a cylindrical outer shape when expanded, and a connecting portion 22 connected to an end portion on the base end side of the cylindrical main body portion 21.

In the expanded state, the cylindrical main body portion 21 has an outer diameter substantially equal to or larger than an inner diameter of the blood vessel V such as the brachiocephalic artery C, and comes into close contact with an inner wall of the blood vessel V so as to press the inner wall of the blood vessel V outward in a radial direction.

The connecting portion 22 has, for instance, a conical shape whose diameter gradually decreases in a direction away from the cylindrical main body portion 21 side in the state where the filter 20 is expanded. In addition, the connecting portion 22 constitutes the base end portion of the filter 20, and a part on the base end side of the connecting portion 22 is connected to the distal end of the sheath tube 10 by an optional method.

As illustrated in FIG. 3, in the filter 20, the cylindrical main body portion 21 and the connecting portion 22 are formed in a mesh shape, and the cylindrical main body portion 21 and the connecting portion 22 can capture the foreign substances F in blood in the blood vessel V. Specifically, the cylindrical main body portion 21 arranged along the inner wall of the blood vessel V captures the foreign substances F flowing along a bloodstream Z from an upstream side to prevent the foreign substances F from diffusing to lateral blood vessels P which should avoid the inflow of the foreign substances F, such as terminal arteries and terminal veins such as the right common carotid artery RCA and the vertebral artery VA in FIG. 1. In addition, the connecting portion 22 captures the foreign substances F which have not been captured in the cylindrical main body portion 21. In such a way, the filter 20 can control the flow of the foreign substances F so that the cylindrical main body portion 21 and the connecting portion 22 capture the foreign substances F.

Incidentally, the cylindrical main body portion 21 and the connecting portion 22 are each made of an elastic material such as metal or a resin, and the materials may be the same or different. Herein, it is preferable to integrally construct the cylindrical main body portion 21 and the connecting portion 22, and thereby the filter 20 can be easily manufactured.

Examples of the methods for releasing and expanding the filter 20 include known methods described in Japanese Patent Laid-Open Publication No. 2000-350785 or WO 2005/99806 brochure, but the literatures are merely examples. The present invention is not limited to the literatures, and the method can be optionally changed as appropriate.

The linear member 30 goes around an opening edge 24 in a distal end portion 23 of the filter 20 and extends along an axial direction of the filter 20 toward the base end side. The linear member 30 is arranged so as to be inserted through the hollow portion 11 of the sheath tube 10.

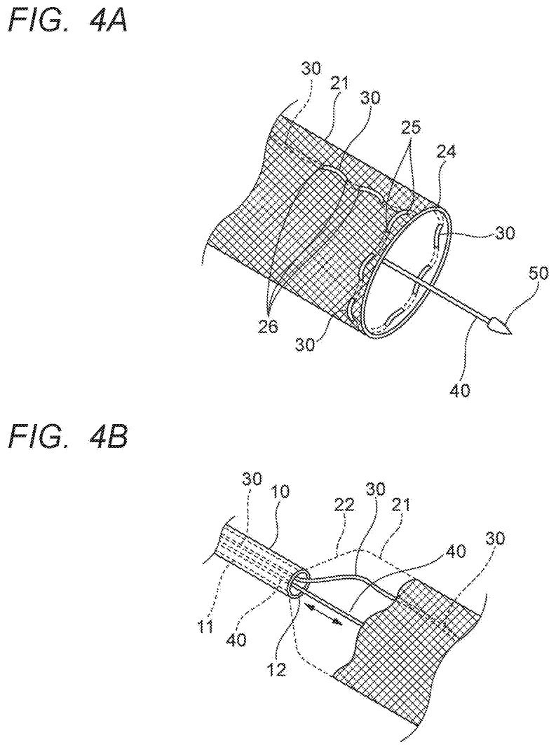

Specifically, as illustrated in FIG. 4A and FIG. 4B, a part on one end side of the linear member 30 is inserted, beginning at a distal end thereof fixed to a specified position on the filter 20 by an optional method, in a plurality of holes 25 formed in the opening edge 24 at intervals in a circumferential direction, to put stitches in the opening edge 24. In addition, the part on one end side of the linear member 30 axially extends and passes through a plurality of holes 26 formed at intervals in an axial direction of the cylindrical main body portion 21, to put stitches in the cylindrical main body portion 21. A part of the linear member 30 that is closer to the base end than the hole 26, which is closest to the base end among the holes 26, is disposed inside the filter 20 (or, the cylinder thereof), and is inserted in the hollow portion 11 through an opening 12 on the distal end side of the sheath tube 10.

Although not illustrated in the figures, the linear member 30 passes through the hollow portion 11 of the sheath tube 10, and a part on the other end side (proximal side) of the linear member 30 is outside the hollow portion 11.

In addition, the linear member 30 constitutes a closing mechanism 61 for closing the opening edge 24 of the distal end portion 23 of the filter 20.

That means, when an end portion on the proximal side (left side in FIG. 5B and the like, for instance) of the linear member 30 is drawn toward the proximal side, the linear member 30 is relatively displaced with respect to the filter 20 to close the opening edge 24 in the distal end portion 23 of the filter 20.

Specifically, by the displacement of the linear member 30, a force for shutting (closing) the opening edge 24 in a gathering manner is applied on the opening edge 24 of the cylindrical main body portion 21, so that the opening edge 24 radially moves inward away from the inner wall of the blood vessel V (e.g., brachiocephalic artery C) where the filter 20 is placed (see FIG. 5B). In other words, the opening edge 24 of the cylindrical main body portion 21 is so deformed as to shut.

Incidentally, the “shutting (closing)” of the opening edge 24 of the cylindrical main body portion 21 means that the cylindrical main body portion 21 is deformed so that an opening area of the opening edge 24 is decreased. Specifically, the cylindrical main body portion 21 may be deformed until the opening area of the opening edge 24 decreases to substantially zero, or until the opening area of the opening edge 24 decreases to a predetermined opening area smaller than the opening area in a state illustrated in FIG. 5A and larger than zero.

In addition, the linear member 30 constitutes a recovery member 60 for recovering the filter 20 placed in the blood vessel V (body lumen) into the sheath tube 10.

That means, in a state where the opening edge 24 of the filter 20 is shut, when the end portion on the proximal side of the linear member 30 is drawn toward the proximal side, the linear member 30 is relatively displaced with respect to the filter 20, so that the filter 20 is recovered by drawing the filter 20 into the sheath tube 10 while turning the distal end portion 23 outside-in.

Specifically, the displacement of the linear member 30 allows the opening edge 24 to be drawn into the cylinder of the filter 20 while turning the distal end portion 23 of the cylindrical main body portion 21 outside-in. At this time, the filter 20 gradually moves away from the inner wall of the brachiocephalic artery C from the distal end side, and then deforms so as to turn inside in the radial direction (see FIG. 5C). Then, when the linear member 30 is further drawn to the proximal side, the filter 20 is drawn into the inside of the hollow portion 11 of the sheath tube 10 from the side of the distal end portion 23 which is turned outside-in, and the whole filter 20 is recovered into the sheath tube 10 (see FIG. 5D).

Incidentally, the linear member 30 is made of any of various materials, such as a resin (plastic, elastomer or the like) and metal, having appropriate hardness and flexibility, but these materials are merely examples. The present invention is not limited to these materials, and the material can be optionally changed as appropriate.

The guide tube 40 is inserted in the hollow portion 11 of the sheath tube 10 and is capable of moving along the axial direction of the sheath tube 10 with respect to the sheath tube 10. Although not illustrated in the figures, an infarction preventive device 1 may be guided to advance and retreat along a guide wire (not illustrated) in the blood vessel V by inserting the guide wire through the guide tube 40.

In addition, the guide tube 40 is made of a resin, a rubber or the like, but these materials are merely examples. The present invention is not limited to these materials, and the material can be optionally changed as appropriate.

The tip 50 has, for instance, such a tapered shape that the diameter gradually decreases from the base end side to the distal end side so that the tip 50 can be percutaneously inserted. A maximum outer diameter of the tip 50 is smaller than an inner diameter of the hollow portion 11 of the sheath tube 10. As illustrated in FIG. 5A and FIG. 5B, when recovering the placed filter 20, the tip 50 is housed together with the guide tube 40 in the hollow portion 11 of the sheath tube 10.

Examples of the material constituting the tip 50 include various materials having appropriate hardness and flexibility, such as a synthetic resin (elastomer) composed of a polyamide-based resin, a polyurethane-based resin, a polyvinyl chloride-based resin, and the like.

Subsequently, a method of using the infarction preventive device 1 will be explained as an example.

In this embodiment, the infarction preventive device 1 is used in combination with, for instance, a stent graft insertion method for placing the stent graft 100 in the aortic arch A, in which the filter 20 of the infarction preventive device 1 is placed in the brachiocephalic artery C (see FIG. 1).

First, an operator percutaneously inserts the infarction preventive device 1 from the inside of the right elbow (not illustrated), and advances the infarction preventive device 1 from a blood downstream side to a target placement position of the brachiocephalic artery C (see Arrow X in FIG. 1). Then, the operator releases the filter 20 from the sheath tube 10 and temporarily place the filter 20 in the brachiocephalic artery C so that the cylindrical main body portion 21 of the radially expanded filter 20 (see FIG. 2) covers branched sites corresponding to the right common carotid artery RCA and the vertebral artery VA.

Then, in order to treat the aortic aneurysm B caused in the aortic arch A different from the brachiocephalic artery C, the operator places the stent graft 100 in the aortic arch A using a known method.

As described above, the infarction preventive device 1 is used in combination with intravascular treatment such as a stent graft-inserting surgery, so that the flow of the foreign substances F in blood in the blood vessel V where the filter 20 is placed can be controlled even when the foreign substances F generated in a site (e.g., aortic arch A) different from the blood vessel V, where the filter 20 of the infarction preventive device 1 is placed, are scattered. Thereby, the foreign substances F can be prevented from flowing into the lateral blood vessels, particularly the terminal arteries and veins not connected to other arteries and veins to properly prevent development of cerebral infarction (necrosis of tissue).

The filter 20 extends during the decrease in diameter and has a network structure in which the foreign substances F get caught up, and therefore can collect the foreign substances F having a size larger than that of a conventional inferior vena cava (IVC) filter or the like without scattering the foreign substances F. In addition, unlike a carotid artery stent protective filter, the filter 20 can be placed from the blood downstream side of the blood vessel V where the filter 20 is to be placed, and therefore the filter 20 can be easily used in combination with another intravascular treatment member that accesses the blood vessel V from the upstream side.

After the stent graft 100 is placed, the operator recovers the filter 20 into the sheath tube 10 according to the procedure illustrated in FIG. 5A to FIG. 5D, for instance, and then removes the entire infarction preventive device 1 from the blood vessel V.

Specifically, as illustrated in FIG. 5A, when the filter 20 is placed in the brachiocephalic artery C, the cylindrical main body portion 21 of the expanded filter 20 covers the branched sites corresponding to the right common carotid artery RCA and the vertebral artery VA. Incidentally, the foreign substances F (see FIG. 3) are captured inside the expanded filter 20.

In such state, first, an end portion on the proximal side (left side in FIG. 5B) of the guide tube 40, which projects from the hollow portion 11 of the sheath tube 10, is drawn toward the proximal side by the operator as illustrated in FIG. 5B, so that the tip 50 is drawn into the hollow portion 11 of the sheath tube 10 and thus recovered together with the guide tube 40. Thereby, the tip 50 (the distal end portion of the infarction preventive device 1) can be prevented from coming into contact with the stent graft 100 placed in the aortic arch A on the central side of the brachiocephalic artery C.

Incidentally, the guide tube 40 may be recovered after the placement of the filter 20, more specifically before the placement of the stent graft 100 in the aortic arch A.

Next, when the operator draws the end portion on the proximal side of the linear member 30 toward the proximal side, a force for shutting the opening edge 24 in a gathering manner is applied onto the opening edge 24 from the linear member 30, so that the opening edge 24 of the cylindrical main body portion 21 is so deformed as to shut.

Subsequently, as illustrated in FIG. 5C, the operator further draws the end portion on the proximal side of the linear member 30 in a state where the opening edge 24 of the cylindrical main body portion 21 is shut. Then the opening edge 24 is drawn into the cylinder of the filter 20 while the distal end portion 23 of the cylindrical main body portion 21 is turned outside-in, and the filter 20 is deformed so as to be gradually turn inward in a radial direction from the distal end side. Furthermore, when the operator further draws the linear member 30 toward the proximal side, the filter 20 is drawn into the inside of the hollow portion 11 of the sheath tube 10 from the distal end portion 23 side where the filter 20 is turned outside-in (see FIG. 5D).

The operator continues to draw the linear member 30 toward the proximal side until the whole filter 20 is recovered inside the hollow portion 11 of the sheath tube 10.

Thereby, the recovery of the filter 20 into the sheath tube 10 is completed.

As described above, the infarction preventive device 1 includes the cylindrical filter 20, the sheath tube 10 for holding the base end portion of the filter 20 and placing the filter 20 in the blood vessel (body lumen) V, and the recovery member 60 for allowing the filter 20 placed in the blood vessel V to be recovered into the sheath tube 10.

Thus, even if not taking the double structure that the shaft connected to the filter is inserted through the hollow portion of the sheath as in the conventional manner, the filter 20 can be recovered into the sheath tube 10 while the foreign substances F captured inside the filter 20 is prevented from re-scattering during recovery of the filter 20. The diameter of the infarction preventive device 1 can be decreased because of unnecessity of the aforementioned double structure.

Since the filter 20 is recovered by drawing the filter 20 into the sheath tube 10 while turning in the distal end portion 23 of the filter 20, that is to say, for instance, the filter 20 is turned outside-in even if foreign substances F such as thrombi adhere to the inner peripheral face of the filter 20, the inner peripheral face side of the filter 20 is not exposed to the inside of the blood vessel V. Accordingly, the foreign substances F can be prevented from re-scattering outside the filter 20. Moreover, the filter 20 can be easily recovered into the sheath tube 10 by a relatively simple operation of displacing the linear member 30 with respect to the sheath tube 10.

In addition, since the opening on the distal end portion 23 of the filter 20 is closed by the closing mechanism 61 during recovery of the filter 20, the foreign substances F captured inside the filter 20 can be prevented from re-scattering outside the filter 20 through the opening of the distal end portion 23. Moreover, the opening of the distal end portion 23 can be easily closed by a relatively simple operation of displacing the linear member 30 with respect to the sheath tube 10.

Furthermore, the filter 20 is housed inside the sheath tube 10 on the blood downstream position of the branched sites of the right common carotid artery RCA and the vertebral artery VA in the brachiocephalic artery C, so that even if a part of the foreign substances F leaking out from gaps of a mesh of the filter 20 scatters during housing of the filter 20, the scattered foreign substances F flow to the blood downstream, and are prevented from flowing into the right common carotid artery RCA and the vertebral artery VA.

Note that the present invention is not limited to the aforementioned embodiments, and modification, improvement, or the like can be applied to the embodiments. In addition, as long as the present invention can be achieved, the materials, shapes, dimensions, numerical values, forms, numbers, placement sites, and the like of the respective constituents in the aforementioned embodiments are arbitrary, and are not limited.

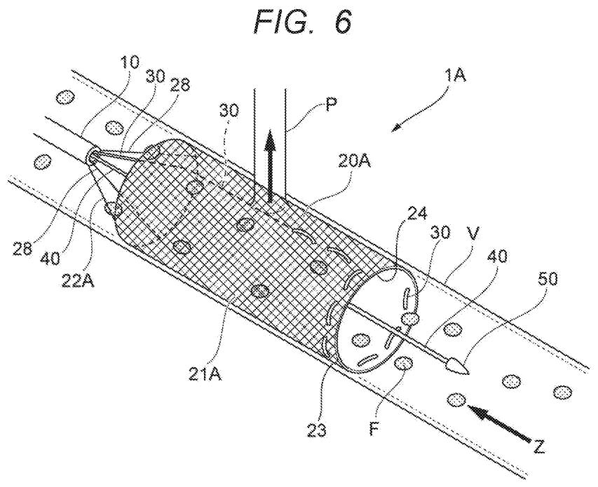

For example, FIG. 6 illustrates a filter 20A of an infarction preventive device 1A according to another embodiment of the present invention. This filter 20A has a structure through which at least a part of the foreign substances F such as thrombi passes.

That is, in the filter 20A, a cylindrical main body portion 21A is formed in a mesh shape, and the foreign substances F in blood in the blood vessel V can be captured. Thereby, the cylindrical main body portion 21A can capture the foreign substances F, but the connecting portion 22A on the most downstream along the bloodstream Z is configured to allow the foreign substances F to pass through. Specifically, the connecting portion 22A is composed of multiple (e.g., four) string-like members 28 for connecting the base end portion of the cylindrical main body portion 21A with the distal end portion of the sheath tube 10.

In this filter 20A, particularly cylindrical main body portion 21A captures the foreign substances F flowing from the upstream side (central side) along the bloodstream Z, and the connecting portion 22A on the most downstream (peripheral side) of the bloodstream Z hardly captures the foreign substances F and is configured to allow the foreign substances F to pass through as much as possible. That means, the flow of the foreign substances F is controlled so that the foreign substances F are intentionally allowed to flow out in an optional direction. This structure has a merit that the foreign substances F can be prevented from scattering during recovery of the filter 20A, a merit that clogging of the filter 20A can be prevented even in a case of much foreign substances F, and the like.

Thus, even if the filter 20A is configured as described above, similarly to the aforementioned embodiments, the filter 20A can be recovered into the sheath tube 10 while preventing the foreign substances F captured inside the filter 20A from re-scattering during housing of the filter 20A without taking the double structure that the shaft connected to the filter is inserted through the hollow portion of the sheath as in the conventional manner. Thereby the diameter of the infarction preventive device 1 can be decreased because of unnecessity of the double structure.

Incidentally, in another embodiment illustrated in FIG. 6, the optional direction for allowing the foreign substances F to flow out is not particularly limited, but basically, examples of the direction include a direction toward a peripheral blood vessel and the like other than the lateral blood vessels P which should avoid the inflow of the foreign substances F, such as the right common carotid artery RCA, the vertebral artery VA, the terminal arteries and the terminal veins.

In addition, the structure of the connecting portion 22A is not limited to the string-like member 28, and may have a mesh shape coarser than that of the cylindrical main body portion 21A. The amount of the foreign substances F to be allowed to flow out or captured can be controlled by adjusting an area of the mesh of the connecting portion 22A, and a size of a space area between the adjacent string-like members 28.

Furthermore, in each of the aforementioned embodiments, one linear member 30 is disposed on the filter 20. However, the number of the linear member 30 is not particularly limited, and multiple (e.g., two) linear members 30 may be disposed on the filter 20. Thereby, when recovering the filter 20 or 20A, closing of the opening of the distal end portion 23 and turning in of the filter 20 or 20A can be more properly performed, and the filter 20 can be properly recovered while preventing the foreign substances F captured inside the filter 20 from re-scattering.

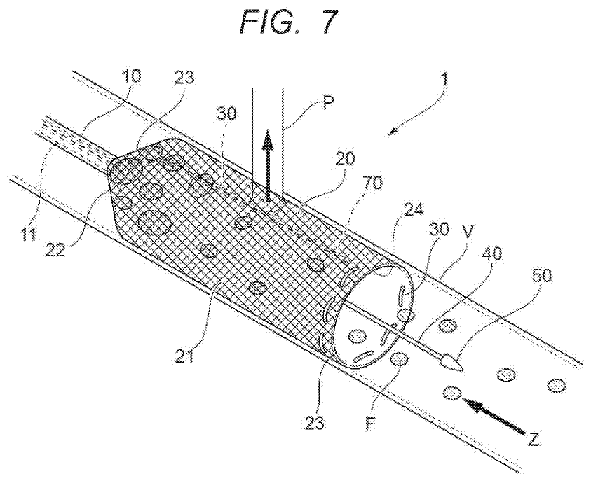

Also, as illustrated in FIG. 7, an auxiliary drawing member 70 may be installed to assist the draw of the filter 20 or 20A into the sheath tube 10. For example, a cylindrical auxiliary drawing member 70 (e.g., flexible tube) which covers the linear member 30 is arranged in the sheath tube 10 and the filter 20 or 20A, and a part on the distal end side of this auxiliary drawing member 70 is previously attached to the distal end portion 23 of the cylindrical main body portion 21 or 21A. First, the linear member 30 is drawn toward the proximal side with respect to the auxiliary drawing member 70, so that the opening edge 24 of the cylindrical main body portion 21 or 21A is shut. Subsequently, the auxiliary drawing member 70 is drawn together with the linear member 30 toward the proximal side so that the cylindrical main body portion 21 or 21A is drawn into the sheath tube 10 while turning in the cylindrical main body portion 21 or 21A from the distal end portion 23 side, and thereby the filter 20 or 20A can be more properly recovered.

In each of the aforementioned embodiments, for instance, a blood vessel other than the one blood vessel V (e.g., brachiocephalic artery C), where the filter 20 or 20A is placed, has been explained as a source region of the foreign substances F, but such blood vessels are merely examples, and the present invention is not limited to these vessels. The source region may be a site different from the site where the filter 20 or 20A is placed in one blood vessel where the filter 20 or 20A is placed, or may be both the site where the filter 20 or 20A is placed, and the site different from the site where the filter 20 or 20A is placed.

The filters 20 and 20A are not necessarily formed in a mesh shape. Although not illustrated in the figures, the filters 20 and 20A may have such a shape that a thin metal wire is bent in a zigzag or wavy shape so that ridge portions and valley portions are alternately formed.

In each of the aforementioned embodiments, the filter 20 or 20A of the infarction preventive device 1 or 1A is placed in the blood vessel V such as brachiocephalic artery C, left common carotid artery LCA, and left subclavian artery LSCA when treating the aortic aneurysm B, but this case is merely an example, and the present invention is not limited to this case. The blood vessel V where the filter 20 or 20A is placed can be optionally changed as appropriate. For example, when treating an abdominal aortic aneurysm (not illustrated), the filters 20 or 20A of the infarction preventive device 1 or 1A may be placed in left and right renal arteries to prevent development of renal infarction. In addition, for example, when treating the blood vessel V such as brachiocephalic artery C, left common carotid artery LCA, and left subclavian artery LSCA, the infarction preventive device 1 or 1A may be placed in the blood vessel V.

From the viewpoint of more properly preventing the foreign substances F from flowing into the right common carotid artery RCA and the vertebral artery VA, the filters 20 and 20A may each be housed in the sheath tube 10 after the entire infarction preventive device 1 including the contracted filter 20 or 20A and the sheath tube 10 is moved to the blood downstream side (left side in FIG. 5D) so as to be away from the branched sites with the right common carotid artery RCA and the vertebral artery VA.

Furthermore, in the aforementioned embodiments, the linear member 30 is inserted through the plurality of holes 25 formed in the vicinity of the opening edge 24 of the cylindrical main body portion 21 or 21A. However, the linear member 30 only needs to be attached to the cylindrical main body portion 21 or 21A so that the opening edge 24 of the cylindrical main body portion 21 or 21A can be shut, and any attachment method may be used. For example, the linear member 30 may be inserted through the inside of the cylindrical portion formed so as to extend along the circumferential direction in the vicinity of the opening edge 24 of the cylindrical main body portion 21 or 21A, or may be spirally formed in the vicinity of the opening edge 24 of the cylindrical main body portion 21 or 21A, or may be formed in a shape bent in a zigzag shape in the circumferential direction.

In the aforementioned embodiment, the filter 20 or 20A has the cylindrical main body portion 21 or 21A and the connecting portion 22 formed in a mesh shape, but this form is merely an example, and the present invention is not limited to this form. The form can be optionally changed as appropriate as long as the shape has an opening through which blood can pass. That means, the shape may be a shape in which a thread-like member (not illustrated) is vertically or horizontally woven, or a shape having a plurality of openings formed by laser processing. Furthermore, the cylindrical main body portion 21 or 21A does not need to entirely come into close contact with the inner wall of the blood vessel V. For example, the inner wall of the blood vessel V is pressed outward in the radial direction and sealed on the distal end portion 23, so that an expanding force of a part other than the distal end portion 23 can be relatively decreased, and a design flexibility of the filter 20 or 20A can be improved.

Furthermore, the infarction preventive device 1 or 1A may be used not only for the purpose of placing and recovering the infarction preventive filter 20 or 20A in the blood vessel V, but as a filter device for placing and recovering the filter in any body lumen such as a digestive tract.

What is claimed is:

1. A filter device comprising:
a filter in a cylindrical form;
a tube configured to hold a base end portion of the filter and place the filter in a body lumen; and
a recovery member configured to recover the filter placed in the body lumen into the tube.

2. The filter device according to claim 1, wherein
the recovery member draws a distal end portion of the filter into the tube while turning the distal end portion outside-in, to recover the filter.

3. The filter device according to claim 2, wherein
the recovery member includes a linear member arranged along an opening edge in the distal end portion of the filter, and
the linear member is displaced with respect to the tube to recover the filter into the tube.

4. The filter device according to claim 2, wherein
the recovery member includes a closing mechanism to close an opening in the distal end portion of the filter.

5. The filter device according to claim 4, wherein
the closing mechanism includes a linear member arranged along an opening edge in the distal end portion of the filter, and
the linear member is displaced with respect to the tube to close the opening in the distal end portion.

6. The filter device according to claim 1, wherein
the recovery member closes an opening in a distal end portion of the filter and draws the distal end portion into the tube while turning the distal end portion outside-in, to recover the filter.
